(19) United States
(12) Patent Application Publication
(10) Pub. No.: US 2025/0213624 A1
(43) Pub. Date: Jul. 3, 2025

(54) INJECTABLE CONNECTIVE TISSUE BIOLOGIC IMPLANT

(71) Applicants: Brian Casey, Pittsburgh, PA (US); Michael Verdon, Dayton, OH (US)

(72) Inventors: Brian Casey, Pittsburgh, PA (US); Michael Verdon, Dayton, OH (US)

(21) Appl. No.: 18/997,862

(22) PCT Filed: Jun. 14, 2022
(86) PCT No.: PCT/US2022/033498

§ 371 Date: Jan. 23, 2025

(51) Int. Cl. A61K 35/50; A61K 31/728; A61L 27/20

(52) U.S. Cl. CPC A61K 35/50; A61K 31/728; A61L 27/20; A61L 2300/64; A61L 2400/06

(57) ABSTRACT

An injectable biologic, along with a method of use, with a viscosity profile capable of remaining in an implant for the duration of a surgery. The injectable biologic can promote bone growth remodeling and repair, along with a method of using the disclosed injectable biologic. The injectable biologic can be used in a variety of surgical procedures, such as spinal interbody implants, long bone fractures, dental procedures, and arthritic conditions and/or as an adjunct for boney healing in, for example, long bone fractures, dental procedures, and/or adapted for use for arthritic conditions.

### CROSS REFERENCE TO RELATED APPLICATIONS

The present application is an international patent application (filed via the Patent Cooperation Treaty).

### BACKGROUND

### 1. Field

The present disclosure relates to the field of spine surgery, specifically to a biologic configured to be injectable without washing out of an implant.

### 2. Discussion of Prior Art

Placement of a biologic on a carrier which is placed in the operative bed or placed into the interbody device. There is no ability for the biologic to interdigitate with a biomechanical device, as a result, most of the biologic “washes out” or leaks out of its intended implant site. This must be done with an instrument or by hand and is not able to be injected through a needle or syringe.

The present invention is directed at overcoming, or at least improving upon, the disadvantages of the prior art.

### BRIEF SUMMARY

The present invention accomplishes this goal by providing an injectable biologic that promotes adherence to an implant's structure, causing the implant to become a biological implant configured to promote bone growth.

The disclosure includes an injectable biologic, which can promote bone growth remodeling and repair, along with a method of using the disclosed injectable biologic.

The disclosure includes a biologic which comprises: a first component comprising a placental allograft; a second component comprising hyaluronic acid; wherein the biologic is configured to be injected; and wherein a ratio by volume of the first component to the second component ranges from 1:1 to 1:10. In the biologic, the hyaluronic acid can comprise a molecular weight of 20,000 to 8×10⁶ KDa. In the biologic, the hyaluronic acid preferably comprises a molecular weight of 65 KDa to 1.8 MDa. In the biologic, the hyaluronic acid can most preferably comprise a molecular weight of 760 to 8,000 KDa. In some embodiments of the biologic, the hyaluronic acid is primarily uncross-linked. In some embodiments of the biologic, the hyaluronic acid comprises a cross-linked range of 1 to 25 percent. In some embodiments of the biologic, the hyaluronic acid comprises sodium hyaluronate. In some embodiments of the biologic, the hyaluronic acid comprises non-animal stabilized hyaluronic acid. In some embodiments of the biologic, the hyaluronic acid comprises sodium acetylated hyaluronate.

The disclosure also includes a method of applying a biologic to an implant, the steps comprising: preparing a biologic configured to be injected by mixing placental allograft with hyaluronic acid in a ratio, by volume, of 1:1 to 1:10; injecting the biologic into voids contained in an implant prior to implantation; and implanting the implant containing the biologic in its voids. In some embodiments of the method, the implant comprises a metallic matrix. In the method, the placental allograft can be in a powdered state. In the method, the hyaluronic acid can comprise a molecular weight of 760 to 8,000 KDa. In some embodiments of the method, the hyaluronic acid is primarily uncross-linked. In some embodiments of the method, the hyaluronic acid comprises a cross-linked range of 1 to 25 percent. In some embodiments of the method, the hyaluronic acid comprises sodium hyaluronate. In some embodiments of the method, the hyaluronic acid comprises non-animal stabilized hyaluronic acid. In some embodiments of the method, the hyaluronic acid comprises sodium acetylated hyaluronate.

### BRIEF DESCRIPTION OF THE DRAWINGS

Many advantages of the present invention will be apparent to those skilled in the art with a reading of this specification in conjunction with the attached drawings, wherein like reference numerals are applied to like elements and wherein:

FIG. 1 is a diagram of the composition of the disclosed injectable biologic;

FIG. 2 is a perspective view of an implant being injected with the disclosed injectable biologic; and

FIG. 3 is a flow chart showing a method of using the disclosed biologic.

### DETAILED DESCRIPTION

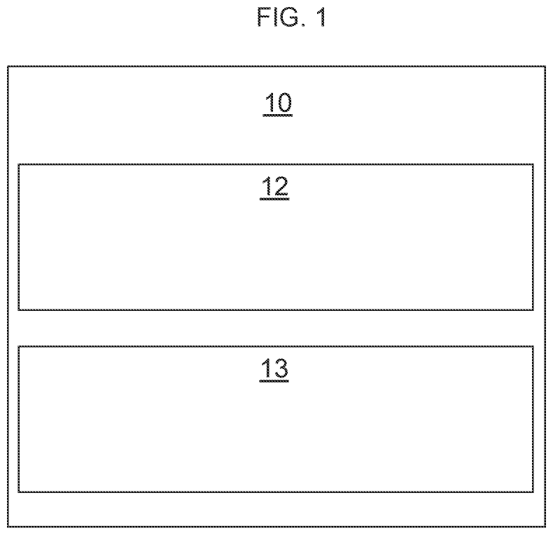

Illustrative embodiments are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure. The leveraged spring clamp disclosed herein boasts a variety of inventive features and components that warrant patent protection, both individually and in combination. In FIG. 1 is a diagram of the composition of the disclosed injectable biologic 10. The injectable biologic 10 has a first component 12 and a second component 13. The first component 12 can be placental allograft and the second component 13 can be hyaluronic acid. In some embodiments, the first component and second component are provided in a ratio (by volume) of 1:1 to 1:10. In the second component 13, the hyaluronic acid can comprise a molecular weight of 20,000 to 8×10⁶ KDa. In the second component 13, the hyaluronic acid preferably comprises a molecular weight of 65 KDa to 1.8 MDa. In the second component 13, the hyaluronic acid most preferably comprises a molecular weight of 760 to 8,000 KDa. In some embodiments of the injectable biologic 10, the hyaluronic acid can be primarily uncross-linked and in some case, can comprise a cross-linked range of 1 to 25 percent. In some embodiments, the hyaluronic acid can comprise sodium hyaluronate, non-animal stabilized hyaluronic acid and/or sodium acetylated hyaluronate.

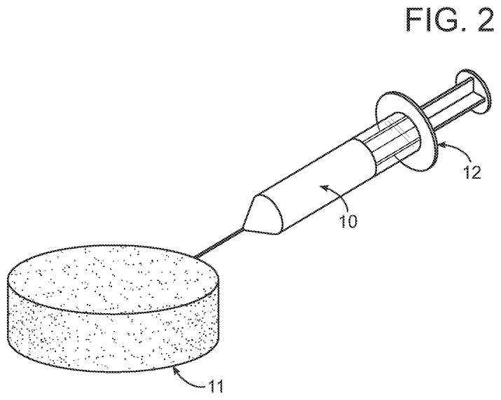

In FIG. 2 is a perspective view of an implant 11 containing at least one void being injected with the disclosed biologic 10.

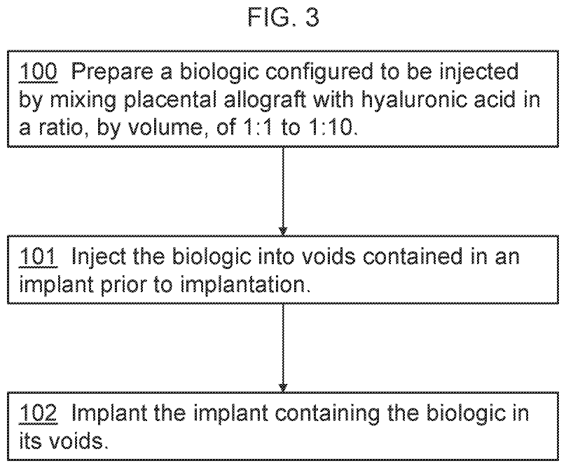

In FIG. 3 is a method of using the disclosed injectable biologic 10 in an implant 11, which can promote bone growth remodeling and repair. The steps listed in the disclosed method may be reordered within the inventive concept and are listed herein in a specific order for clarity. In step 100, prepare a biologic configured to be injected by mixing placental allograft with hyaluronic acid in a ratio, by volume, of 1:1 to 1:10. In a step 101, inject the biologic into voids contained in an implant prior to implantation. In a step 102, implant the implant 11 containing the biologic 10 in its voids. In some embodiments of the disclosed method, the implant 11 can comprise a metallic matric structure. In some embodiments of the disclosed method, the placental allograft can be in a powdered state. In some embodiments of the disclosed method, the hyaluronic acid can have a molecular weight of 760 to 8,000 KDa. The hyaluronic acid can also be primarily uncross-linked and/or comprise a cross-linked range of 1 to 25 percent. In some embodiments, the hyaluronic acid can be sodium hyaluronate, non-animal stabilized hyaluronic acid, and/or sodium acetylated hyaluronate.

What has been described is an injectable biologic 10 with a viscosity profile capable of remaining in implants throughout the operation and recovery. In this disclosure, there are shown and described only exemplary embodiments of the invention, but, as aforementioned, it is to be understood that the invention is capable of use in various other combinations and environments and is capable of changes or modifications within the scope of the inventive concept as expressed herein.

From the foregoing disclosure and detailed description of certain preferred embodiments, it is also apparent that various modifications, additions and other alternative embodiments are possible without departing from the true scope and spirit. The embodiments discussed were chosen and described to provide the best illustration of the principles of the present invention and its practical application to thereby enable one of ordinary skill in the art to utilize the invention in various embodiments and with various modifications as are suited to the particular use contemplated. All such modifications and variations are within the scope of the present invention as determined by the appended claims when interpreted in accordance with the benefit to which they are fairly, legally, and equitably entitled.

What is claimed is:

1. A biologic which comprises:
a first component comprising a placental allograft;
a second component comprising hyaluronic acid;
wherein the biologic is configured to be injected; and
wherein a ratio by volume of the first component to the second component ranges from 1:1 to 1:10.

2. The biologic of claim 1, wherein the hyaluronic acid comprises a molecular weight of 20,000 to 8×10⁶ KDa.

3. The biologic of claim 1, wherein the hyaluronic acid comprises a molecular weight of 65 KDa to 1.8 MDa.

4. The biologic of claim 1, wherein the hyaluronic acid comprises a molecular weight of 760 to 8,000 KDa.

5. The biologic of claim 1, wherein the hyaluronic acid is primarily uncross-linked.

6. The biologic of claim 1, wherein the hyaluronic acid comprises a cross-linked range of 1 to 25 percent.

7. The biologic of claim 1, wherein the hyaluronic acid comprises sodium hyaluronate.

8. The biologic of claim 1, wherein the hyaluronic acid comprises non-animal stabilized hyaluronic acid.

9. The biologic of claim 1, wherein the hyaluronic acid comprises sodium acetylated hyaluronate.

10. A method of applying a biologic to an implant, the steps comprising:
preparing a biologic configured to be injected by mixing placental allograft with hyaluronic acid in a ratio, by volume, of 1:1 to 1:10;
injecting the biologic into voids contained in an implant prior to implantation; and
implanting the implant containing the biologic in its voids.

11. The method of claim 10, wherein the implant comprises a metallic matrix.

12. The method of claim 10, wherein the placental allograft is in a powdered state.

13. The method of claim 10, wherein the hyaluronic acid comprises a molecular weight of 760 to 8,000 KDa.

14. The method of claim 10, wherein the hyaluronic acid is primarily uncross-linked.

15. The method of claim 10, wherein the hyaluronic acid comprises a cross-linked range of 1 to 25 percent.

16. The method of claim 10, wherein the hyaluronic acid comprises sodium hyaluronate.

17. The method of claim 10, wherein the hyaluronic acid comprises non-animal stabilized hyaluronic acid.

18. The method of claim 10, wherein the hyaluronic acid comprises sodium acetylated hyaluronate.
